(19) United States
(12) Patent Application Publication
(10) Pub. No.: US 2013/0217934 A1
(43) Pub. Date: Aug. 22, 2013

(54) PROCESS FOR PRODUCING AROMATIC HYDROCARBONS AND ETHYLENE

(71) Applicants: Leslie Andrew Chewter, Amsterdam (NL); Hervé Henry, Rotterdam (NL); Ajay Madgavkar, Katy, TX (US); Jeroen Van Westrenen, Amsterdam (NL)

(72) Inventors: Leslie Andrew Chewter, Amsterdam (NL); Hervé Henry, Rotterdam (NL); Ajay Madgavkar, Katy, TX (US); Jeroen Van Westrenen, Amsterdam (NL)

(21) Appl. No.: 13/642,152

(22) PCT Filed: Apr. 19, 2011
(86) PCT No.: PCT/EP2011/056200

§ 371 Date: Dec. 18, 2012

Related U.S. Application Data
(60) Provisional application No. 61/327,350, filed on Apr. 23, 2010.

(51) Int. Cl. C07C 2/76; C07C 1/02; C07C 2/64

(52) U.S. Cl. CPC C07C 2/76; C07C 2/64; C07C 1/02
USPC 585/303; 585/304; 422/620

(57) ABSTRACT

The present invention provides a process for producing aromatic hydrocarbons and ethylene, comprising: a. contacting a lower alkane feed comprising at least one of ethane, propane and butane with an aromatic hydrocarbon conversion catalyst within an alkane-to-aromatic zone to obtain at least hydrogen and aromatic reaction products, including at least benzene; b. converting an oxygenate feedstock in an oxygenate-to-olefin zone to obtain olefins, including at least ethylene; wherein at least part of the oxygenate feedstock is obtained by providing at least part of the hydrogen obtained in step a) and a feed containing carbon monoxide and/or carbon dioxide to an oxygenate synthesis zone and synthesizing oxygenates. In another aspect the invention provides an integrated system for aromatic hydrocarbons and ethylene and the use of hydrogen obtained from a process to convert lower alkanes to benzene to produce an oxygenate feed for an oxygenate-to-olefin process.

### FIELD OF THE INVENTION

The invention relates to a process for producing aromatic hydrocarbons and ethylene and an integrated system for producing ethylene and benzene.

### BACKGROUND OF THE INVENTION

In recent years increasing attention is given to the exploration and utilisation of natural gas resources around the globe. A disadvantage of natural gas with respect to oil is the difficulty to transport large volumes of natural gas from the source to the market. One way of efficiently transporting natural gas is by liquefying the natural gas and to transport the liquefied natural gas (LNG). Another way is to convert the methane in the natural gas to liquid hydrocarbons using a Gas-to-Liquid process (GtL). The GtL products are typically liquid and can be transported in a similar way as traditional oil and oil products.

Besides methane, the natural gas typically comprises other hydrocarbons such as ethane, propane, and butanes. Such a natural gas is referred to as wet gas. Ethane can be thermally cracked into ethylene, which is a base chemical for producing e.g. polyethylene, styrene, ethylene oxide or mono-ethyl-glycol. The propane and butanes can be added to the LPG pool.

In case the natural gas is withdrawn from relatively small reservoirs, especially those located in remote isolate locations, also referred to as stranded natural gas, the reservoirs cannot achieve sufficient production levels to sustain a GtL or LNG plant. In addition, insufficient ethane is co-produced to sustain an ethane to ethylene process and subsequent ethylene conversion processes. It has been suggested to combine an Oxygenate-to-Olefin (OTO) processes with an ethane steam cracker. In such a combination the methane is converted to ethylene and propylene in the OTO process, while the ethane is cracked to produce additional ethylene. For instance, C. Eng et al. (C. Eng, E. Arnold, E Vora, T. Fuglerud, S. Kvisle, H. Nilsen, Integration of the UOP/HYDRO MTO Process into Ethylene plants, 10^{th} Ethylene Producers' Conference, New Orleans, USA, 1998) have suggested to combine UOP's Methanol-to-Olefins (MTO) process with a naphtha or ethane fed steam cracker. It is mentioned that by combining both processes sufficient ethylene can be produced, while coproducing valuable propylene. A disadvantage mentioned by C. Eng et al. is the fluctuating price of methanol, which is the primary feed to the MTO reaction.

Methanol can be produced from hydrogen and carbon monoxide or carbon dioxide. Typically, methanol is produced from a mixture of hydrogen, carbon monoxide and carbon dioxide.

In WO 2009/039948 A2, a combined steam cracking and MTP process is suggested for preparing ethylene and propylene. According to WO 2009/039948 A2, in this process, a particular advantage is obtained by combining the back-end of both processes. The methanol feedstock is produced from methane, requiring a sufficient supply of methane.

In order to synthesize methanol, hydrogen, carbon monoxide and carbon dioxide should be provided in a molar ratio of at least 2, which ratio is calculated by:

molar ratio=(#mol H₂−#mol CO₂)/(#mol CO+#mol CO₂).

The feed to a methanol synthesis is typically a synthesis gas. However, such a synthesis gas of course needs contain hydrogen, carbon monoxide and carbon dioxide in a molar ratio of at least 2. Most exothermic synthesis gas processes, however, produce a synthesis gas that is hydrogen deficient. It is not sufficient to for instance pass the hydrogen deficient synthesis gas to a water-gas-shift reactor to convert part of the carbon monoxide in the synthesis gas with water to hydrogen and carbon dioxide. As can be seen in the definition of the molar ratio herein above, such a conversion does not influence the molar ratio obtained.

In US20050038304, an integrated system for producing ethylene and propylene from an OTO system and a steam cracking system is disclosed. According to US20050038304, in this process, a particular advantage is obtained by combining the back-end of both processes. The methanol feedstock to the OTO process is produced from synthesis gas. However, according to US20050038304 the production of methanol from synthesis gas has high energy requirements due to the endothermic nature of the synthesis gas production process, such an endothermic synthesis gas production process is normally steam methane reforming.

The conversion of ethane to ethylene is highly endothermic and requires significant energy input. In addition, the ethane must first be separated from the propane and butane, prior to feeding the ethane to the ethane steam cracker. As a result the capex and opex for the ethane to ethylene conversion are even further increased, while the obtained LPG is only of limited economic value for reasons of economy of scale and size limitations due to the limited availability of LPG.

There is a need in the art for an improved process for converting C1 to C4 alkanes (also known as paraffins) to valuable products.

### SUMMARY OF THE INVENTION

It has now been found that it is possible to produce aromatic hydrocarbons and ethylene from C1 to C4 alkanes by aromatisation of C2 to C4 alkanes to aromatic hydrocarbons and converting Cl to methanol, which is used as methanol feedstock to an Oxygenate to Olefin (OTO) process. In this process, hydrogen obtained from the aromatisation process is used to produce at least part of the methanol feed to the OTO process.

Accordingly, the present invention provides a process for producing aromatic hydrocarbons and ethylene, comprising:

a. contacting a lower alkane feed comprising at least one of ethane, propane and butane with an aromatic hydrocarbon conversion catalyst within an alkane-to-aromatic zone to obtain at least hydrogen and aromatic reaction products including at least benzene;
b. converting an oxygenate feedstock in an oxygenate-to-olefin zone to obtain olefins including at least ethylene;

wherein at least part of the oxygenate feedstock is obtained by providing at least part of the hydrogen obtained in step a) and a feed containing carbon monoxide and/or carbon dioxide to an oxygenate synthesis zone and synthesizing oxygenates.

The process according to the invention converts ethane and less valuable LPG into valuable aromatic hydrocarbons, such as benzene, in an alkane to aromatic process (further also referred to as ATA process). There is a projected global shortage for benzene which is needed in the manufacture of key petrochemicals such as styrene, phenol, nylon and polyurethanes, among others. Presently, benzene and other aromatic hydrocarbons are obtained by separating a feedstock fraction, which is rich in aromatic compounds, such as reformate produced through a catalytic reforming process and pyrolysis gasolines produced through a naphtha cracking process, from non-aromatic hydrocarbons using a solvent extraction process or extractive distillation process.

Furthermore, the process according to the invention converts methane into valuable ethylene using an Oxygenate to Olefin process (further also referred to as OTO process). The ethylene may subsequently be used to produce further valuable chemical products.

In addition, the process according to the invention provides a synergy between an ATA process and an OTO process by using the hydrogen obtained by converting lower alkanes, including at least one of ethane, propane and butane, into aromatic hydrocarbons in the ATA process, to produce at least part of the oxygenate feedstock to the OTO process. The hydrogen obtained from the ATA process is further also referred to as hydrogen ex. ATA. As a result, less hydrogen needs to be supplied through other means, such as an endothermic synthesis gas producing process like Steam Methane Reforming (SMR). As a result, the carbon dioxide penalty for producing oxygenates is reduced as at least part of the hydrogen required for producing the oxygenate is obtained as co-product.

The process according to the present invention is directed at producing aromatic hydrocarbons and ethylene. In a preferred embodiment the process is directed at producing ethylbenzene. In a further preferred embodiment the process is directed at producing styrene monomer.

### BRIEF DESCRIPTION OF THE DRAWINGS

FIG. 1, a schematic representation is given of an embodiment of integrated system for producing olefins according to the invention.

### DETAILED DESCRIPTION OF THE INVENTION

In step (a) of the process according to the present invention, a lower alkane feed comprising at least one of ethane, propane and butane is provided. This feed is further also referred to as lower alkane feed. The lower alkane feed is provided to an ATA zone and contacted with an aromatic hydrocarbon conversion catalyst under aromatic conversion conditions in an ATA process. At least part of the ethane, propane and/or butane, and optionally pentane and/or hexane if present, in the lower alkane feed is converted in the ATA zone to aromatic hydrocarbon products. In contact with the catalyst, the ethane, propane and/or butane may be converted to several aromatic conversion products. One of the obtained aromatic conversion products is benzene, however other aromatic conversion products such as toluene and xylenes, including m-xylene, p-xylene and o-xylene, may also be obtained. An additional product obtained for the ATA process is hydrogen. In theory and depending on the exact reactants and reaction anywhere between 2 to 6 moles of hydrogen (H₂) may be produced per mole of benzene formed. The ATA process may produce other hydrocarbon by-products such as for instance methane.

As mentioned herein above, other aromatic conversion products, in particular toluene and xylenes may be produced by the ATA process in the ATA zone. It is preferred that at least part of the toluene and xylenes produced are converted to benzene. Preferably, toluene and xylenes are converted to benzene by a hydrodealkylation process in the presence of hydrogen to obtain further benzene and methane.

In step (b) of the process, an oxygenate feed is provided to an oxygenate-to-olefin zone and converted to obtain olefins including at least ethylene. Preferably, the OTO process produces, in addition to ethylene, also propylene. The oxygenate feedstock may comprise any oxygenate or mixture of oxygenates. Preferred oxygenates include alkylalcohols and alkylethers, more preferably methanol, ethanol, propanol and/or dimethylether (DME), even more preferably methanol and/or dimethylether (DME).

A synergy is achieved in the process according to the invention by using at least part of the hydrogen obtained in step (a), i.e. hydrogen ex. ATA, to produce at least part of the oxygenate feed which is provided to the OTO zone in step (b). As a result, the hydrogen produced during the ATA process is no longer disposed of, for instance as combustion fuel in a furnace, but rather used to produce valuable oxygenates. In addition, the hydrogen obtained from step (a) does not comprise significant amounts of inerts such as N₂ and Ar. These inerts may typically be present in the natural gas or purified oxygen provided to produce synthesis gas for methanol production. By providing hydrogen obtained from step (a) as part of the feed to the oxygenate synthesis zone the inert content in this feed may be reduced.

Further synergy is achieved as the process according to the invention allows the use of mixed feedstocks, e.g. a primarily C1 to C4 alkane comprising feedstock, to produce aromatic hydrocarbons and ethylene. In such a case, the feedstock is split into a stream comprising predominantly C2 to C4 alkanes, which is converted to at least benzene, and a stream comprising predominantly methane, which is converted to synthesis gas and subsequently methanol and/or DME. The methanol and/or DME can be converted to at least ethylene using an OTO process.

As mentioned herein above, hydrogen obtained in step (a) is used to produce at least part of the oxygenate feedstock provided to the OTO zone in step (b).

Any suitable oxygenate or mixture of oxygenates may be produced, in particular alkylalcohols and alkylethers, preferably methanol and/or DME.

In the process according to the invention, hydrogen and a feed containing carbon monoxide and/or carbon dioxide are provided to an oxygenate synthesis zone.

Methanol may be produced directly from hydrogen and at least one of carbon monoxide and carbon dioxide in the oxygenate synthesis zone. Hydrogen can react with carbon monoxide to produce methanol following:

CO+2H₂→CH₃OH.

Alternatively hydrogen may react with carbon dioxide to also form methanol following:

CO₂+3H₂→CH₃OH+H₂O.

It is also possible to use a mixture of carbon monoxide and carbon dioxide. Preferably, the hydrogen and carbon monoxide and/or carbon dioxide are provided to the oxygenate synthesis zone in a molar ratio of in the range of from 1.6 to 3.0, preferably 2.0 to 3.0, more preferably 2.0 to 2.2. The molar ratio herein is defined as:

molar ratio=(#mol H₂−#mol CO₂)/(#mol CO+#mol CO₂).

In the above definition at least one of the number of moles carbon monoxide or the number of moles carbon dioxide is higher than zero. As the stoichiometric molar ratio is 2.0, not all to the carbon monoxide and/or carbon dioxide will react, in case a molar ratio below 2.0 is used.

In case a mixture of carbon monoxide and carbon dioxide is used to convert the hydrogen to methanol, it is preferred that the carbon dioxide concentration in the hydrogen, carbon monoxide and carbon dioxide mixture is in the range of from 0.1 to 25 mol %, preferably 3 to 15 mol %, more preferably of from 4 to 10 mol %, based on the total number of moles hydrogen, carbon monoxide and carbon dioxide in the mixture. The carbon dioxide content, relative to that of CO, in the syngas should be high enough so as to maintain an appropriately high reaction temperature and rate and to minimize the amount of undesirable by-products such as paraffins and higher alcohols. At the same time, the relative carbon dioxide to carbon monoxide content should not be too high so as the reaction of carbon dioxide with hydrogen yields less methanol based on the hydrogen provided to the oxygenate synthesis zone. In addition, the reaction of carbon dioxide with hydrogen yields water. If present in too high concentration, water may deactivate the oxygenate synthesis catalyst.

In the oxygenate synthesis zone, the hydrogen and carbon monoxide and/or carbon dioxide are converted to methanol in the presence of a suitable catalyst. Such catalysts are known in the art and are for instance described in WO 2006/020083, which is incorporated herein by reference. Suitable catalyst for the synthesis of methanol from hydrogen and at least one of carbon monoxide and carbon dioxide include:
An oxide of at least one element selected from the group consisting of copper, silver, zinc, boron, magnesium, aluminium, vanadium, chromium, manganese, gallium, palladium, osmium and zirconium.
Preferably, the catalyst is a copper and zinc based catalyst, more preferably in the form of copper, copper oxide, and zinc oxide.
A copper based catalyst, which includes an oxide of at least one element selected from the group consisting of silver, zinc, boron, magnesium, aluminium, vanadium, chromium, manganese, gallium, palladium, osmium and zirconium.
Preferably, the catalyst contains copper oxide and an oxide of at least one element selected from the group consisting of zinc, magnesium, aluminium, chromium, and zirconium.
A catalyst selected from the group consisting of: copper oxides, zinc oxides and aluminium oxides. More preferably, the catalyst contains oxides of copper and zinc.
A catalyst comprising copper oxide, zinc oxide, and at least one other oxide.
Preferably, the at least one other oxide is selected from the group consisting of zirconium oxide, chromium oxide, vanadium oxide, magnesium oxide, aluminium oxide, titanium oxide, hafnium oxide, molybdenum oxide, tungsten oxide, and manganese oxide.

Particular suitable catalysts include catalysts comprising in the range of from 10 to 70 wt % copper oxide, based on total weight of the catalyst. Preferably, comprising in the range of from 15 to 68 wt % copper oxide, and more preferably of from 20 to 65 wt % copper oxide, based on total weight of the catalyst.

Such catalyst may preferably also contain in the range of from 3 to 30 wt % zinc oxide, based on total weight of the catalyst. Preferably, contain in the range of from 4 to 27 wt % zinc oxide, more preferably of from 5 to 24 wt % zinc oxide, based on total weight of the catalyst.

Catalyst comprising both copper oxide and zinc oxide, preferably comprise copper oxide and zinc oxide in a ratio of copper oxide to zinc oxide which may vary over a wide range. Preferably, such catalyst comprise copper oxide to zinc oxide in a Cu:Zn atomic ratio in the range of from 0.5:1 to 20:1, preferably of from 0.7:1 to 15:1, more preferably of from 0.8:1 to 5:1.

The catalyst can be prepared according to conventional processes. Examples of such processes can be found in US6114279; US6054497; US5767039; US5045520; US5254520; US5610202; US4666945; US4455394; US4565803; and US5385949, with the descriptions of each being fully incorporated herein by reference.

Methanol may be synthesised in the oxygenate synthesis zone by any conventional methanol synthesis process. Examples of such processes include batch processes and continuous processes. Continuous processes are preferred.

Tubular bed processes and fluidized bed processes are particularly preferred types of continuous processes.

The methanol synthesis process is effective over a wide range of temperatures. Preferably, methanol is synthesised in the oxygenate synthesis zone by contacting the hydrogen and at least one of one of carbon monoxide and carbon dioxide with the catalyst at a temperature of in the range of from 150 to 450° C., more preferably of from 175 to 350° C., even more preferably of from 200 to 300° C.

The methanol synthesis process is effective over a wide range of pressures. Preferably, the methanol is synthesised by contacting the hydrogen and at least one of carbon monoxide and carbon dioxide with the catalyst in the oxygenate synthesis zone at a pressure of in the range of from 15 to 125 atmospheres, more preferably of from 20 to 100 atmospheres, more preferably of from 25 to 75 atmospheres. Preferably, at least part hydrogen obtained in step (a) and/or at least part of the feed containing carbon monoxide and/or carbon dioxide is pressurised prior to providing the hydrogen and feed containing carbon monoxide and/or carbon dioxide to the oxygenate synthesis zone.

For methanol synthesis, gas hourly space velocities in the oxygenate synthesis zone vary depending upon the type of continuous process that is used. Preferably, gas hourly space velocity of flow of gas through the catalyst bed is in the range of from 50 hr⁻¹ to 50,000 hr⁻¹. Preferably, gas hourly space velocity of flow of gas through the catalyst bed is in the range of from about 250 hr⁻¹ to 25,000 hr⁻¹, more preferably from about 500 hr⁻¹ to 10,000 hr⁻¹.

A methanol synthesis process as described herein above may produce several oxygenates as by-products, including aldehydes and other alcohols. Such by-products are also suitable reactants in the OTO reaction. Other less desirable by-products may be removed from the effluent of the oxygenate synthesis zone effluent if required prior to providing the oxygenate synthesis zone effluent to the OTO zone as to form at least part of the oxygenate feed.

Another suitable and preferred oxygenate, which may be synthesised in the oxygenate synthesis zone is dimethylether (DME). DME can be directly synthesized from hydrogen obtained in step (a) and at least one of carbon monoxide and carbon dioxide, but is preferably synthesized from methanol, which was at least in part produced from hydrogen obtained in step (a) as described herein above. Optionally, DME is obtained from methanol and hydrogen and at least one of carbon monoxide and carbon dioxide. The conversion of methanol to DME is known in the art. This conversion is an equilibrium reaction. In the conversion the alcohol is contacted at elevated temperature with a catalyst. In EP-A 340 576, a list of potential catalysts are described. These catalysts include the chlorides of iron, copper, tin, manganese and aluminum, and the sulphates of copper, chromium and aluminum. Also oxides of titanium, aluminum or barium can be used. Preferred catalysts include aluminum oxides and aluminum silicates. Alumina is particularly preferred as catalyst, especially gamma-alumina. Although the methanol may be in the liquid phase the process is preferably carried out such that the methanol is in the vapour phase. In this context the reaction is suitably carried out at a temperature of 140 to 500° C., preferably 200 to 400° C., and a pressure of 1 to 50 bar, preferably from 8-12 bar, the exact choice depends on the acidity of the catalyst. In view of the exothermic nature of the conversion of methanol to DME the conversion is suitably carried out whilst the reaction mixture comprising the catalyst is being cooled to maximize DME yield.

Suitably, the methanol to DME reaction takes place in a separate section of the oxygenate synthesis zone.

In case, part of the methanol synthesized is converted into DME, the effluent of the oxygenate zone may comprise methanol and DME in any ratio. Preferably, the ratio of DME to methanol weight ratio of in the range of from 0.5:1 to 100:1, more preferably from 2:1 to 20:1. Suitably the methanol to DME conversion reaction is reaction is led to equilibrium. This includes that the DME to methanol weight ratio may vary from 2:1 to 6:1. Evidently, the skilled person may decide to influence the equilibrium by applying different reaction conditions and/or by adding or withdrawing any of the reactants.

Preferably, in the process according to the invention at least part of the oxygenate feed is methanol and/or DME, produced by reacting hydrogen obtained from step (a) with at least one of carbon monoxide and carbon dioxide.

The feed containing carbon monoxide and/or carbon dioxide may be any feed containing carbon monoxide and/or carbon dioxide available. A particularly suitable feed containing carbon monoxide and/or carbon dioxide is feed comprising a synthesis gas obtained from a process for preparing synthesis gas. Such processes for preparing synthesis gas preferably include non-catalytic partial oxidation processes, catalytic partial oxidation processes, steam methane reforming processes, auto-thermal reforming processes, and water-gas-shift processes. Although, a water-gas-shift process is in principle not a process for preparing a synthesis gas, the effluent of a water-gas-shift process typically comprises hydrogen, carbon monoxide, and carbon dioxide. The feed may also comprise synthesis gas obtained from several processes for preparing synthesis gas.

Preferred sources of carbon monoxide and/or carbon dioxide are those that comprise synthesis gas having a hydrogen and carbon monoxide and/or carbon dioxide molar ratio, as defined herein above, which is below the ratio preferred for synthesising methanol, i.e. sources that are hydrogen deficient. Such synthesis gases are typically obtained from synthesis gas producing processes, in which natural gas or another methane-comprising gas is partially oxidised to provide synthesis gas feed for a Fischer-Tropsch process. Such processes for preparing synthesis gas preferably include non-catalytic partial oxidation processes, catalytic partial oxidation processes and auto-thermal reforming processes.

Preferably, the synthesis gas provided as the feed containing carbon monoxide and/or carbon dioxide has a molar ratio of hydrogen to carbon monoxide and/or carbon dioxide of in the range of from 1.0 to 1.9, more preferably of from 1.3 to 1.8, wherein the molar ratio is defined as herein above. Such low carbon dioxide synthesis gases are preferably produced by non catalytic partial oxidation processes for preparing synthesis gas. A reforming catalyst typically induces some water-gas-shift in the presence of water. As a result, carbon monoxide is shifted to carbon dioxide. An additional advantage is that non-catalytic partial oxidation processes do not require the addition of substantial amounts of water to the process. Processes producing substantial amounts of carbon dioxide include for instance Steam Methane Reforming. Therefore, the use of a synthesis gas from a Steam Methane Reforming process is less preferred.

The process according to the invention includes embodiments wherein the hydrogen obtained in step (a) is provided to and/or mixed with an effluent of a syngas producing process and subsequently the at least part of the effluent, optionally after being processed in a water-gas-shift step, is used for the oxygenate synthesis process.

The use of a part of a synthesis gas stream, of which the remainder is used as a feed to a Fischer-Tropsch process, has the additional advantage that carbon dioxide in the synthesis gas stream can predominantly be directed towards the oxygenate synthesis process rather than to the Fischer-Tropsch process in which carbon dioxide is regarded as an undesired inert.

As mentioned herein above, one of the by products of step (a) can be methane. This methane may be obtained directly from the conversion of the lower alkane feed to aromatic hydrocarbons or from the hydrodealkylation of any produced toluene or xylenes. Preferably, at least part of the methane produced in step (a) is converted to syngas using one of the above mentioned processes for preparing synthesis gas. Optionally, the methane is added to the feed to an existing process for preparing synthesis gas.

The synthesis gas produced from the methane obtained from step (a) can be provided to the oxygenate synthesis zone to produce further oxygenate feedstock.

Another suitable feed containing carbon monoxide and/or carbon dioxide is a feed comprising carbon dioxide obtained from a subsurface natural gas or oil reservoir. Such carbon dioxide is also referred to as field carbon dioxide. Some subsurface natural gas or oil reservoirs comprise substantial concentration of carbon dioxide, up to 70 mol % based on the total gas volume extracted from the reservoir. By using this carbon dioxide to synthesise oxygenates and subsequently olefins, this carbon dioxide is captured, reducing the carbon dioxide penalty for exploiting the subsurface natural gas or oil reservoir.

Another suitable feed containing carbon monoxide and/or carbon dioxide is a source comprising carbon dioxide obtained from a carbon dioxide-comprising flue gas stream, in particular a flue gas obtained from the integrated process according to the invention or optionally an oxygen purification unit or synthesis gas production process. Preferably, the flue gas is first concentrated to increase the carbon dioxide concentration.

A particularly suitable feed containing carbon monoxide and/or carbon dioxide may be a feed comprising carbon dioxide obtained from a process for preparing ethylene oxide or optionally Mono-ethylene-Glycol (MEG).

Another particular suitable feed containing carbon monoxide and/or carbon dioxide may be a feed comprising carbon dioxide obtained from regeneration of the aromatic hydrocarbon conversion catalyst. The catalyst becomes deactivated by coke formation. The coke is periodically removed, for instance by oxidation. In particular when the oxidation of the coke on the catalyst is done using pure oxygen or a mixture of oxygen and carbon dioxide an almost pure stream of carbon dioxide and optionally carbon monoxide can be obtained.

As mentioned herein above it is preferred to use a feed containing carbon monoxide and/or carbon dioxide that comprises both carbon monoxide and carbon dioxide, therefore preferably a synthesis gas is combined with at least one stream comprising carbon dioxide to form the feed containing carbon monoxide and/or carbon dioxide. For example, a synthesis gas comprising mainly hydrogen and carbon monoxide may be combined with field carbon dioxide to form a feed containing carbon monoxide and/or carbon dioxide, which can be mixed with at least part of the hydrogen obtained in step (a). Preferably, sufficient carbon dioxide is added to the synthesis gas to provide a carbon dioxide concentration in the range of from 0.1 to 25 mol %, preferably 3 to 15 mol %, more preferably of from 4 to 10 mol %, based on the total number of moles hydrogen, carbon monoxide and carbon dioxide in the mixture.

Preferably, a synthesis gas is used comprising little or no carbon dioxide. The carbon dioxide from e.g. a MEG process or catalyst decoking comprises little or no inerts like Ar, or N₂. When using a synthesis gas with little or no carbon dioxide, more carbon dioxide from for instance a MEG process can be added and less inerts are introduced to the oxygenate synthesis zone. Less waste carbon dioxide is thus produced, which would otherwise need to be sequestrated or captured and stored.

In step (a) of the process, hydrogen is produced together with aromatic hydrocarbons, typically hydrogen and aromatic hydrocarbons leave the ATA zone as an ATA zone effluent comprising hydrogen and aromatic hydrocarbons. Preferably, the hydrogen is separated from aromatic hydrocarbons, i.e. the ATA zone effluent comprising hydrogen and aromatic hydrocarbons, prior to being provided to the oxygenate synthesis zone. The hydrogen may be separated using any suitable means known in the art, for example cryogenic distillation, pressure swing adsorption whereby impurities in the hydrogen containing stream absorb preferentially over the hydrogen or via hydrogen permeable membrane. Preferably, a pressure swing adsorption process is used to separate the hydrogen from the remainder of the stream.

In step (b) of the process according to the present invention the oxygenate feedstock is converted and the reaction products leave the OTO zone as an OTO zone effluent. In addition to the desired olefins, such as ethylene and propylene, also some by-products are obtained, including alkanes, higher olefins and aromatics. These are preferably separated from the desired ethylene and propylene in the OTO zone effluent. Preferably, a fraction comprising C5+ hydrocarbons, i.e. hydrocarbons comprising 5 or more carbon atoms, and more preferably a fraction comprising C5 to C9 hydrocarbons, is separated from the OTO zone effluent. This fraction may comprise C5+ alkanes, C5+ olefins and C5+ aromatics, and preferably comprises C5 to C9 alkanes, C5 to C9 olefins and C5 to C9 aromatics. This fraction may be hydrogenated to saturate at least part and preferably all of the olefins in the fraction. Preferably, this fraction is selectively hydrogenated to hydrogenate the olefins, but not the aromatics. Subsequent to the hydrogenation, or preferably selective hydrogenation, the hydrogenated fraction may be provided to the ATA zone, as part of or separate from the lower alkane feed. Any aromatics in the hydrogenated fraction will pass through the ATA zone unconverted, while the alkanes, including cyclo-alkanes will be converted to aromatics. This is particularly advantageous as the aromatic content in OTO zone effluent is generally insufficient to warrant a dedicated aromatic removal unit provided only to separate the low amounts of aromatics from the OTO zone effluent. By adding the C5+ hydrocarbon fraction, or preferably C5 to C9 hydrocarbon fraction, to ATA zone, the aromatics are separated together with the aromatics produced in the ATA zone. In addition, by providing the C5+ alkanes and hydrogenated C5+ olefins to the ATA zone, additional hydrogen is produced as at least part of the C5+ alkanes and hydrogenated C5+ olefins are converted to aromatics in the ATA zone. As a result, not only the aromatics yield is increased, but also more olefins may be produced in step (b) by using the hydrogen to produce further oxygenates for use in step (b). At least part of the hydrogen produced in step (a) may be used to hydrogenate at least part of the C5+ fraction.

Alternatively, an aromatic fraction, preferably comprising benzene, may be separated from the OTO zone effluent and combined with at least part of the ATA zone effluent.

Step (b) of the process may also produce small amounts of lower alkanes, in particular ethane, propane and butane as a by-product. A further synergy of the process can be obtained by providing any ethane, propane and/or butane present in the effluent of the OTO zone to the ATA zone. The ethane, propane and butane can then be converted to aromatic hydrocarbons and hydrogen in the ATA zone, thus providing additional aromatic hydrocarbons and hydrogen. The hydrogen may subsequently be used to synthesise oxygenates.

The ethylene produced in step (b) of the process according to the invention can be used as a feedstock for several other processes to produce chemical products, including the production of ethylene oxide, mono-ethyl-glycol (MEG), ethylbenzene and styrene monomer.

It has now also been found that it is possible to integrate the production of these products into the process according to the invention to obtain further synergy.

In one embodiment of the invention, the process is a process for preparing ethylbenzene. In this embodiment a further integration is provided by addition of a further step (c), comprising reacting at least part of the benzene obtained in step (a) with at least part of the ethylene obtain in step (b) to obtain ethylbenzene. The reaction of benzene with ethylene is well known in the art. Any suitable process may be used. Ethyl benzene is typically produced by reacting ethylene and benzene in the presence of an acid catalyst. Reference is for example made to Kniel et al., Ethylene, Keystone to the petrochemical industry, Marcel Dekker, Inc, New York, 1980, in particular section 3.4.1, page 24 to 25.

Ethylbenzene is a valuable chemical product. It is an organic chemical compound, which is an aromatic hydrocarbon. Its major use is in the petrochemical industry as an intermediate compound for the production of styrene, which in turn is used for making polystyrene, a commonly used plastic material. Methods for the reaction of benzene with ethylene to produce ethylbenzene are described in US20090156870, which is herein incorporated by reference in its entirety.

In addition, contrary to ethylene, ethyl benzene is a liquid with a boiling point above the boiling point of benzene. As a result, ethyl benzene more easily stored and transported compared to ethylene or even benzene. Typically, the produced ethylene is used as a base-chemical for the production of polyethylene. A particular advantage of converting at least part of the ethylene with benzene to ethylbenzene is that there is no need to provide polymer-grade ethylene, i.e. very high purity, for the reaction between ethylene and benzene, thereby reducing extent to which the ethylene needs to be purified following the OTO reaction step.

In another embodiment of the invention, the process is a process for preparing styrene monomer. In this embodiment a further integration is provided, by addition of a further step (d) and (e), comprising

(d) dehydrogenating ethylbenzene obtained in step (c) to obtain styrene monomer and hydrogen, and

(e) providing hydrogen obtained in step (d) to the oxygenate synthesis zone to synthesize oxygenates. The oxygenates can be used as part of the oxygenate feedstock to the OTO zone in step (b).

Styrene monomer is produced by the catalytic dehydrogenation of ethylbenzene. Typically, the dehydrogenation is performed at elevated temperatures, preferably in the range of from 500 to 700° C. The dehydrogenation typically takes place in the presence of steam, preferably at a steam to ethylbenzene molar ratio of in the range of from 1 to 30, more preferably of from 2 to 20. The catalyst may be any suitable catalyst. Examples of suitable catalyst include but are not limited to dehydrogenation catalysts based on iron(III) oxide. Optionally, the catalyst may include promoters such as rare earth metals or calcium typically in the form of oxides or carbonates. One process for producing styrene is described in US4857498, which is herein incorporated by reference in its entirety. Another process for producing styrene is described in US7276636, which is herein incorporated by reference in its entirety.

By integrating the process according to the invention with the production of styrene monomer, as described above, further hydrogen is produced. Preferably, this hydrogen is separated and subsequently provided to the oxygenate synthesis zone to prepare at least part of the oxygenate feedstock to step (b) of the process.

By using hydrogen obtained from the conversion of ethylene and benzene, via ethylbenzene, into styrene to synthesise oxygenates, the carbon dioxide penalty for producing oxygenates is reduced as at least part of the hydrogen required for producing the oxygenate is obtained as co-product and does not add additional carbon dioxide on top of what is required for the main reaction product styrene monomer.

The produced styrene monomer may be used to produce polystyrene.

Preferably, at least part of the ethylene produced in step (b) is oxidised to ethylene oxide by providing at least part of the ethylene with a source of oxygen to an ethylene oxidation zone, further referred to as EO zone.

Preferably, the ethylene oxide is further converted to mono-ethylene-glycol (MEG). MEG is a liquid and therefore can be transported and stored more conveniently than ethylene oxide. Preferably, the EO zone is part of a larger Mono-ethylene-glycol synthesis zone, i.e. a second oxygenate synthesis zone, further referred to as MEG zone. Preferably, the MEG zone then comprises a first section comprising the EO zone and a second ethylene oxide hydrolyses section. The MEG is synthesised by providing the ethylene oxide with a source of water to the ethylene oxide hydrolyse zone and converting the ethylene oxide to MEG. Optionally, the ethylene oxide is first reacted with carbon dioxide to from ethylene carbonate, which is subsequently hydrolysed to obtain MEG and carbon dioxide, reference herein is made to for instance US2008139853, incorporated by reference.

Ethylene is typically converted to ethylene oxide by oxidising ethylene to form ethylene oxide.

The conversion of ethylene to ethylene oxide may be done by any ethylene oxidation process that produces at least ethylene oxide and carbon dioxide. In the EO zone, at least a part of the ethylene is partly oxidised to form ethylene oxide. Preferably, the oxidation of ethylene takes place in the EO zone to which the ethylene and a source of oxygen are provided. Preferably, the source of oxygen is oxygen-enriched air or, more preferably pure oxygen. The oxidation of ethylene may be performed over a catalyst present in the first section, preferably a silver based catalyst. Reference is for example made to Kniel et al., Ethylene, Keystone to the petrochemical industry, Marcel Dekker, Inc, New York, 1980, in particular page 20. As a by-product of the oxidation of ethylene to ethylene oxide carbon dioxide is formed. Without wishing to be bound to any theory, the production of carbon dioxide is believed to originate from a reaction of ethylene with catalyst bound oxygen atoms. As a consequence in the range of from 14 to 20 mol % of the total amount of ethylene provided to the EO zone is converted into carbon dioxide. As for the conversion of ethylene with benzene to ethyl benzene described herein above, there is no need to provide polymer-grade ethylene to produce the ethylene oxide, thereby reducing the extent to which the ethylene needs to be purified.

The conversion of ethylene oxide to MEG may be done using any MEG producing process that uses ethylene oxide. Typically the ethylene oxide is hydrolysed with water to MEG. Optionally, the ethylene oxide is first converted with carbon dioxide to ethylene carbonate, which is subsequently hydrolysed to MEG and carbon dioxide. The water is provided to the MEG zone as a source of water, preferably pure water or steam. The MEG product is obtained from the MEG zone as a MEG-comprising effluent. Suitable processes for the production of ethylene oxide and MEG are described for instance in US2008139853, US2009234144, US2004225138, US20044224841 and US2008182999, herein incorporated by reference, however any suitable process for producing ethylene oxide and converting the ethylene oxide to MEG may be used.

As mentioned, a by-product of the ethylene oxide/MEG process is carbon dioxide. During the oxidation reaction of ethylene to ethylene oxide, carbon dioxide is formed. This is waste carbon dioxide and needs to be sequestered or otherwise captured and stored. In the process according to the present invention this carbon dioxide may be used to form at least part of the feed containing carbon monoxide and/or carbon dioxide provided to the oxygenate synthesis zone.

Preferably, the carbon dioxide is separated from the OE zone effluent to obtain a separate carbon dioxide comprising stream. Preferably, the EO zone effluent is further treated to convert the ethylene oxide into MEG in a MEG zone. From the MEG zone a MEG zone effluent is obtained, comprising MEG and optionally carbon dioxide. Suitably, the carbon dioxide can be separated from the MEG zone effluent by cooling the MEG zone effluent to a temperature below the boiling point of MEG, this carbon dioxide is also referred to as carbon dioxide ex. MEG. As no additional carbon dioxide is produced by converting ethylene oxide into MEG the carbon dioxide ex. MEG is the same as the carbon dioxide ex. EO. By reusing the carbon dioxide to synthesize oxygenates, the carbon dioxide penalty for producing EO is reduced. A further advantage is that the stream comprising carbon dioxide obtained from the EO or MEG zone comprises predominantly carbon dioxide and, depending on the temperature of the stream, steam. Preferably the stream comprises in the range of 80 to 100 mol % of carbon dioxide and steam, based on the total amount of moles in the stream. More preferably, the stream comprising carbon dioxide comprises essentially only carbon dioxide and, optionally, steam. Such a stream is particularly suitable to be used in an oxygenate synthesis process as it does not introduce significant amounts of inert, e.g. CH₄, N₂ and Ar, to the oxygenate synthesis zone. Should, however, the stream comprising carbon dioxide comprise significant amounts of other, undesired, compounds, e.g. ethylene oxide, the stream is preferably treated to remove such compounds prior to being introduced into the oxygenate synthesis zone. Another advantage of the integration with a MEG synthesis is that next to MEG minor amount of other oxygenates may produced in the MEG zone by the process for producing MEG. These oxygenates may suitably be separated from the obtained MEG zone effluent and provided to the OTO zone as part of the oxygenate feed.

A preferred process according to the invention could comprise:
i) providing a feed comprising methane and at least one of ethane, propane and butane;
ii) separating the feed into at least a methane-comprising feed and a lower alkane feed, comprising at least one of ethane, propane and butane;
iii) providing at least part the methane-comprising feed to a process for preparing synthesis gas to obtain a synthesis gas; and
iv) contacting the lower alkane feed with an aromatic hydrocarbon conversion catalyst in an alkane-to-aromatic zone according to step (a) to obtain at least benzene and hydrogen;
v) providing at least part of the hydrogen obtained in step iv) and at least part the synthesis gas obtained in step iii) to an oxygenate synthesis zone and synthesising oxygenates; and
vi) converting at least part of the oxygenates in an oxygenate-to-olefin zone according to step (b) to obtain at least ethylene.

Optionally, next to hydrogen and synthesis gas, also carbon dioxide is provided to the oxygenate synthesis zone in (v), i.e. additional to the carbon dioxide that may already be comprised in the synthesis gas.

Preferably, the oxygenates are synthesised in step (v) by converting the hydrogen with carbon monoxide and/or carbon dioxide into at least methanol and/or dimethylether. The embodiment of the process according to the invention allows for the co-production of aromatic hydrocarbons and ethylene from a feed comprising methane and at least one of ethane, propane and butane, such as for example a natural gas or associated gas. Reference herein to associated gas is to C1 to C5 hydrocarbons co-produced with the production of oil.

The synthesis of oxygenates may be done in the direct vicinity of the OTO zone, or alternatively the oxygenate synthesis and the oxygenate conversion to at least ethylene may take place at different locations, while the oxygenate is transported from the location where the oxygenate is synthesised to the location where the oxygenate is converted. Preferably, the oxygenate is transported together with at least part of the benzene produced in step (a), in case at least part of the benzene is to be reacted to ethylbenzene and/or styrene monomer with at least part of the ethylene produced by the conversion the oxygenate.

In the process according to the invention more oxygenate may be synthesised than required for the conversion of oxygenate to olefins in step (b). In that case, at least part of the oxygenates synthesised may be exported from the process to be used for other purposes, such as the production of MTBE (methyl tert-butyl ether), TAME(tert-amyl ether) if the oxygenate is methanol, or as a feed to another OTO process.

As mentioned herein above, step (a) of the process according to the invention is a process for producing aromatic hydrocarbons from a lower alkane feed comprising at least one of ethane, propane and butane, which comprises bringing into contact the lower alkane feed with an aromatic hydrocarbon conversion catalyst composition suitable for promoting the reaction of such alkanes to aromatic hydrocarbons, such as benzene, at a temperature in the range of from 400 to about 700° C., preferably of from 450° C. to 660° C. and a pressure of from about 0.01 to about 1.0 MPa absolute. The gas hourly space velocity (GHSV) per hour may range from about 300 to about 6000. The primary products of the process of this invention are at least benzene, but typically often include toluene and xylenes.

It is possible to carry out this process in batch mode or in continuous mode. The ATA zone may comprise single reactor or in two or more reactors aligned in parallel. Preferably, at least two reactors are used so that one reactor may be in use for aromatization while the other reactor is offline so the catalyst may be regenerated. The aromatization reactor system may be a fluidized bed, moving bed or a cyclic fixed bed design. The cyclic fixed bed design is preferred for use in this invention.

Any one of a variety of catalysts may be used to promote the reaction of ethane, propane and/or butane and possibly other alkanes to aromatic hydrocarbons. One such catalyst is described in US4899006, which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP0244162, which is herein incorporated by reference in its entirety. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in US7186871 and US7186872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminium-free.

It is preferred that the catalyst be comprised of a zeolite, a noble metal of the platinum family to promote the dehydrogenation reaction, and a second inert or less active metal which will attenuate the tendency of the noble metal to catalyze hydrogenolysis of the C2 and higher alkanes in the feed to methane and/or ethane. Attenuating metals which can be used include those described below.

Additional catalysts which may be used in the process of the present invention include those described in US 5227557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in US20090209795, hereby incorporated by reference in its entirety. This application describes a catalyst comprising: (1) 0.005 to 0.1% wt (% by weight) platinum, based on the metal, preferably 0.01 to 0.05% wt, (2) an amount of an attenuating metal selected from the group consisting of tin, lead, and germanium which is preferably not more than 0.2% wt of the catalyst, based on the metal and wherein the amount of platinum may be no more than 0.02% wt more than the amount of the attenuating metal; (3) 10 to 99.9% wt of an aluminosilicate, preferably a zeolite, based on the aluminosilicate, preferably 30 to 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is a catalyst comprising: (1) 0.005 to 0.1% wt (% by weight) platinum, based on the metal, preferably 0.01 to 0.06% wt, most preferably 0.01 to 0.05% wt, (2) an amount of iron which is equal to or greater than the amount of the platinum but not more than 0.50% wt of the catalyst, preferably not more than 0.20% wt of the catalyst, most preferably not more than 0.10% wt of the catalyst, based on the metal; (3) 10 to 99.9% wt of an aluminosilicate, preferably a zeolite, based on the aluminosilicate, preferably 30 to 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in US 20090209794, hereby incorporated by reference in its entirety. This application describes a catalyst comprising: (1) 0.005 to 0.1 wt % (% by weight) platinum, based on the metal, preferably 0.01 to 0.05% wt, most preferably 0.02 to 0.05% wt, (2) an amount of gallium which is equal to or greater than the amount of the platinum, preferably no more than 1 wt %, most preferably no more than 0.5 wt %, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

One of the undesirable products of the aromatization reaction is coke which may deactivate the catalyst. While catalysts and operating conditions and reactors are chosen to minimize the production of coke, it is usually necessary to regenerate the catalyst at some time during its useful life. Regeneration may increase the useful life of the catalyst.

Regeneration of coked catalysts has been practiced commercially for decades and various regeneration methods are known to those skilled in the art.

The regeneration of the catalyst may be carried out in the aromatization reactor or in a separate regeneration vessel or reactor. For example, the catalyst may be regenerated by burning the coke at high temperature in the presence of an oxygen-containing gas as described in US4795845 which is herein incorporated by reference in its entirety. Regeneration with air and nitrogen is shown in the examples of US4613716 which is herein incorporated by reference in its entirety. Another possible method involves air calcination, hydrogen reduction, and treatment with sulphur or a sulphurization material. Platinum catalysts have been used to assist the combustion of coke deposited on such catalysts.

The preferred regeneration temperature range for use herein is from about 450 to about 790° C. The preferred temperature range for regeneration in the first stage is from about 470 to about 790° C. The preferred temperature range for regeneration in the second stage is from about 500 to about 790° C.

Toluene and xylenes may be converted into benzene by hydrodealkylation. The hydrodealkylation reaction involves the reaction of toluene, xylenes, and higher aromatics with hydrogen to strip alkyl groups from the aromatic ring to produce additional benzene and light ends including methane and ethane which are separated from the benzene. This step substantially increases the overall yield of benzene and thus is highly advantageous.

Both thermal and catalytic hydrodealkylation processes are known in the art. Methods for hydrodealkylation are described in US20090156870, which is herein incorporated by reference in its entirety.

Preferably, part of the hydrogen obtained from the process for converting alkanes to aromatic hydrocarbon conversion products is used to provide hydrogen required to the hydrodealkylation of toluene and/or xylenes.

The methane may be used for any other purpose such as a fuel for a furnace. Preferably it is used to produce additional synthesis gas as described herein above. If ethane is present in ATA zone effluent, either because part of the ethane in the feed past through the ATA zone unreacted or produced as a by-product of the hydrodealkylation of toluene or xylenes, this ethane can be recycled to the ATA zone.

In the present invention, an oxygenate feedstock is converted in an oxygenate-to-olefins process, in which an oxygenate feedstock is contacted in an OTO zone with an oxygenate conversion catalyst under oxygenate conversion conditions, to obtain a conversion effluent comprising lower olefins. In the OTO zone, at least part of the feed is converted into a product containing one or more olefins, preferably including lower olefins, in particular ethylene and typically propylene.

Examples of oxygenates that can be used in the oxygenate feedstock of step b) of the process include alcohols and ethers. Examples of preferred oxygenates include alcohols, such as methanol, ethanol, propanol; and dialkyl ethers, such as dimethylether, diethylether, methylethylether.

The oxygenate used in the process according to the invention is preferably an oxygenate which comprises at least one oxygen-bonded alkyl group. The alkyl group preferably is a C1-C5 alkyl group, more preferably C1-C4 alkyl group, i.e. comprises 1 to 5, respectively, 4 carbon atoms; more preferably the alkyl group comprises 1 or 2 carbon atoms and most preferably one carbon atom. The oxygenate may preferably comprise one or more of such oxygen-bonded C1-C5 alkyl groups, more preferably C1-C4 alkyl groups. Preferably, the oxygenate comprises one or two oxygen-bonded C1-C5 alkyl groups, more preferably C1-C4 alkyl groups.

More preferably an oxygenate is used having at least one C1 or C2 alkyl group, still more preferably at least one C1 alkyl group.

Preferably, the oxygenate is chosen from the group of alkanols and dialkyl ethers consisting of dimethylether, diethylether, methylethylether, methanol, ethanol and isopropanol, and mixtures thereof.

Most preferably the oxygenate is methanol or dimethylether, or a mixture thereof.

Preferably the oxygenate feedstock comprises at least 50 wt % of oxygenate, in particular methanol and/or dimethylether, based on total hydrocarbons, more preferably at least 70 wt %, even more preferably 80 wt %, most preferably at least 90 wt %.

The oxygenate feedstock can be obtained from a prereactor, which converts methanol at least partially into dimethylether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of diluents, such as water or steam.

A variety of OTO processes is known for converting oxygenates such as for instance methanol or dimethylether to an olefin-containing product, as already referred to above. One such process is described in WO-A 2006/020083, incorporated herein by reference, in particular in paragraphs [0116]-[0135]. Processes integrating the production of oxygenates from synthesis gas and their conversion to light olefins are described in US20070203380A1 and US20070155999A1.

Catalysts as described in WO A 2006/020083 are suitable for converting the oxygenate feedstock in step (b) of the present invention. Such catalysts preferably include molecular sieve catalyst compositions. Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56.

Alternatively, the conversion of the oxygenate feedstock may be accomplished by the use of an aluminosilicate catalyst, in particular a zeolite. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48. Aluminosilicate catalysts are preferred when an olefinic co-feed is fed to the oxygenate conversion zone together with oxygenate, for increased production of ethylene and propylene.

The reaction conditions of the oxygenate conversion include those that are mentioned in WO-A 2006/020083. Hence, a reaction temperature of 200 to 1000° C., preferably from 250 to 750° C., and a pressure from 0.1 kPa (1 mbar) to 5 MPa (50 bar), preferably from 100 kPa (1 bar) to 1.5 MPa (15 bar), are suitable reaction conditions.

A specially preferred OTO process for use in step (b) of the present invention will now be described. This process provides particularly high conversion of oxygenate feed and a recycle co-feed to ethylene and propylene. Reference is made in this regard also to WO2007/135052, WO2009/065848, WO2009/065875, WO2009/065870, WO2009/065855, WO2009/065877, in which processes a catalyst comprising an aluminosilicate or zeolite having one-dimensional 10-membered ring channels, and an olefinic co-feed and/or recycle feed is employed.

In this process, the oxygenate-conversion catalyst comprises one or more zeolites having one-dimensional 10-membered ring channels, which are not intersected by other channels, preferably at least 50% wt of such zeolites based on total zeolites in the catalyst. Preferred examples are zeolites of the MTT and/or TON type. In a particularly preferred embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11. Such further zeolite (molecular sieve) can have a beneficial effect on the stability of the catalyst in the course of the OTO process and under hydrothermal conditions. The second molecular sieve having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80, more preferably at least 100, even more preferably at least 150. The catalyst may comprise phosphorous as such or in a compound, i.e. phosphorous other than any phosphorous included in the framework of the molecular sieve. It is preferred that an MEL or MFI-type zeolites comprising catalyst additionally comprises phosphorous. The phosphorous may be introduced by pre-treating the MEL or MFI-type zeolites prior to formulating the catalyst and/or by post-treating the formulated catalyst comprising the MEL or MFI-type zeolites. Preferably, a catalyst comprising MEL or MFI-type zeolites comprises phosphorous as such or in a compound in an elemental amount of from 0.05-10 wt % based on the weight of the formulated catalyst.

The oxygenate conversion catalyst can comprise at least 1 wt %, based on total molecular sieve in the oxygenate conversion catalyst, of the second molecular sieve having more-dimensional channels, preferably at least 5 wt %, more preferably at least 8 wt %.

Especially when the oxygenate conversion is carried out over a catalyst containing MTT or TON type aluminosilicates, it may be advantageous to add an olefin-containing co-feed together with the oxygenate feed (such as dimethylether-rich or methanol-rich) feed to the OTO zone when the latter feed is introduced into this zone. It has been found that the catalytic conversion of the oxygenates, in particular methanol and DME, to ethylene and propylene is enhanced when an olefin is present in the contact between methanol and/or dimethylether and the catalyst. Therefore, suitably, an olefinic co-feed is added to the reaction zone together with the oxygenate feedstock.

In special embodiments, at least 70 wt % of the olefinic co-feed, during normal operation, is formed by a recycle stream of a C3+ or C4+ olefinic fraction from the OTO conversion effluent, preferably at least 90 wt %, more preferably at least 99 wt %, and most preferably the olefinic co-feed is during normal operation formed by such recycle stream. In one embodiment the olefinic co-feed can comprise at least 25, preferably at least 50 wt %, of C4 olefins, and at least a total of 70 wt % of C4 hydrocarbon species. It can also comprise propylene. The OTO conversion effluent can comprise 10 wt % or less, preferably 5 wt % or less, more preferably 2 wt % or less, even more preferably 1 wt % or less of C6-C8 aromatics, based on total hydrocarbons in the effluent. At least one of the olefinic co-feed, and the recycle stream, can in particular comprise less than 20 wt % of C5+ olefins, preferably less than 10 wt % of C5+ olefins, based on total hydrocarbons in the olefinic co-feed.

In order to maximize production of ethylene and propylene, it is desirable to maximize the recycle of C4 olefins. In a stand-alone process, i.e. without integration with an ATA process, there is a limit to the maximum recycle of a C4 fraction from the OTO effluent. A certain part thereof, such as between 1 and 5 wt %, needs to be withdrawn as purge, since otherwise saturated C4's (butane) would build up which are substantially not converted under the OTO reaction conditions. As mentioned herein above, alkanes in the OTO zone effluent are preferably provided to the ATA zone in step (a) to be converted into aromatic hydrocarbon conversion products. Optionally, the part of the purge that is provided to the ATA zone is first hydrogenated to saturate any unsaturated hydrocarbons.

In the preferred process, optimum light olefins yields are obtained when the OTO conversion is conducted at a temperature of more than 450° C., preferably at a temperature of 460° C. or higher, more preferably at a temperature of 480° C. or higher, in particular at 500° C. or higher, more in particular 550° C. or higher, or 570° C. or higher. The temperature will typically less than 700° C., or less than 650° C. The pressure will typically be between 0.5 and 15 bar, in particular between 1 and 5 bar.

In a special embodiment, the oxygenate conversion catalyst comprises 50 wt %, preferably more than 50 wt %, more preferably at least 65 wt %, based on total molecular sieve in the oxygenate conversion catalyst, of the one-dimensional molecular sieve having 10-membered ring channels.

In one embodiment, molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. When the molecular sieves are prepared in the presence of organic cations the molecular sieve may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 500° C. for 1 hour or more. The molecular sieves obtained after ion-exchange are also referred to as being in the ammonium form.

The molecular sieve can be used as such or in a formulation, such as in a mixture or combination with a so-called binder material and/or a filler material, and optionally also with an active matrix component. Other components can also be present in the formulation. If one or more molecular sieves are used as such, in particular when no binder, filler, or active matrix material is used, the molecular sieve itself is/are referred to as oxygenate conversion catalyst. In a formulation, the molecular sieve in combination with the other components of the mixture such as binder and/or filler material is/are referred to as oxygenate conversion catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the molecular sieve is therefore incorporated in a binder material. Examples of suitable materials in a formulation include active and inactive materials and synthetic or naturally occurring zeolites as well as inorganic materials such as clays, silica, alumina, silica-alumina, titania, zirconia and aluminosilicate. For present purposes, inactive materials of a low acidity, such as silica, are preferred because they may prevent unwanted side reactions which may take place in case a more acidic material, such as alumina or silica-alumina is used.

Typically the oxygenate conversion catalyst deactivates in the course of the process. Conventional catalyst regeneration techniques can be employed. The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

The preferred embodiment of step (b) described hereinabove is preferably performed in an OTO zone comprising a fluidized bed or moving bed, e.g. a fast fluidized bed or a riser reactor system, although in general for an OTO process, in particular for an MTP process, also a fixed bed reactor or a tubular reactor can be used. Serial reactor systems can be employed.

In one embodiment, the OTO zone comprises a plurality of sequential reaction sections. Oxygenate can be added to at least two of the sequential reaction sections.

When multiple reaction zones are employed, an olefinic co-feed is advantageously added to the part of the dimethylether-rich feed that is passed to the first reaction zone.

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

A diluent can also be fed to the OTO zone, mixed with the oxygenate and/or co-feed if present, or separately. A preferred diluents is steam, although other inert diluents can be used as well. In one embodiment, the molar ratio of oxygenate to diluent is between 10:1 and 1:10, preferably between 4:1 and 1:2, most preferably between 3:1 and 1:1, such as 2:1 or 1.5:1, in particular when the oxygenate is methanol and the diluent is water (steam).

The lower alkane feed comprising at least ethane, propane and butane may be any suitable lower alkane feed comprising at least ethane, propane and butane. Reference herein to a lower alkane is to alkanes having in the range of from 2 to 10 carbon atoms. Reference herein to a lower alkane feed is to a hydrocarbonaceous feed comprising at least one alkane having in the range of from 2 to 10 carbon atoms. Preferably, the lower alkane feed comprises at least ethane and propane. More preferably, the lower alkane feed comprises at least 20% wt of ethane, at least 20% wt of propane, and, optionally, at least about 10 to 20% wt of butane and higher alkanes, based on the total weight of the lower alkane feed. Even more preferably, the lower alkane feed comprises in the range of from 30 to 50 wt % ethane and in the range of from 30 to 50 wt % ethane, based on the total weight of the lower alkane feed. The lower alkane feed may contain small amounts of C₂-C₄ olefins, preferably no more than 5 to 10 wt %, based on the total weight of the lower alkane feed. Too much olefin may cause an unacceptable amount of coking and deactivation of the catalyst.

A lower alkane feed may be, for example, an ethane/propane-rich stream derived from natural gas, refinery or petrochemical streams including waste streams. Examples of potentially suitable feed streams include (but are not limited to) residual ethane and propane and butane from natural gas (methane) purification, pure ethane and propane and butane streams (also known as Natural Gas Liquids) co-produced at a liquefied natural gas (LNG) site, C₂-C₄ streams from associated gases co-produced with crude oil production (which are usually too small to justify building a LNG plant but may be sufficient for a chemical plant), unreacted “waste” streams from steam crackers, and the C₁-C₄ byproduct stream from naphtha reformers (the latter two are of low value in some markets such as the Middle East). Another suitable feed stream may be a stream comprising C2 to C4 paraffins obtained from a OTO process.

The oxygenate feedstock provided to step (b) of the first process for producing olefins according to the invention may be any oxygenate-comprising feedstock. The oxygenate feedstock comprises at least an oxygenate, preferably methanol and/or DME, obtained by providing hydrogen ex. ATA and a feed containing carbon monoxide and/or carbon dioxide to an oxygenate synthesis zone and converting hydrogen with carbon monoxide and/or carbon dioxide into preferably methanol and/or dimethylether. The oxygenate feedstock may further comprise oxygenates, such as for example other alcohols, other ethers, aldehydes, ketones and esters. Preferably, the oxygenate feedstock comprises water as a diluent. The oxygenate feedstock may also comprise compounds other than water and oxygenates.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

The oxygenate feedstock may provided directly from one or more oxygenate synthesis zones, however, it may also be provided from a central oxygenate storage facility.

The olefinic co-feed optionally provided together with the oxygenate feedstock to the OTO conversion zone may contain one olefin or a mixture of olefins. Apart from olefins, the olefinic co-feed may contain other hydrocarbon compounds, such as for example paraffinic, alkylaromatic, aromatic compounds or a mixture thereof. Preferably, the olefinic co-feed comprises an olefinic fraction of more than 20 wt %, more preferably more than 25 wt %, still more preferably more than 50 wt %, which olefinic fraction consists of olefin(s). The olefinic co-feed can consist essentially of olefin(s).

Any non-olefinic compounds in the olefinic co-feed are preferably paraffinic compounds. If the olefinic co-feed contains any non-olefinic hydrocarbon, these are preferably paraffinic compounds. Such paraffinic compounds are preferably present in an amount in the range from 0 to 80 wt %, more preferably in the range from 0 to 75 wt %, still more preferably in the range from 0 to 50 wt %.

By an unsaturate is understood an organic compound containing at least two carbon atoms connected by a double or triple bond. By an olefin is understood an organic compound containing at least two carbon atoms connected by a double bond. The olefin can be a mono-olefin, having one double bond, or a poly-olefin, having two or more double bonds. Preferably olefins present in an olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferred olefins have in the range from 2 to 12, preferably in the range from 3 to 10, and more preferably in the range from 4 to 8 carbon atoms.

Examples of suitable olefins that may be contained in the olefinic co-feed include ethene, propene, butene (one or more of 1-butene, 2-butene, and/or iso-butene (2-methyl-1-propene)), pentene (one or more of 1-pentene, 2-pentene, 2-methyl-1-butene, 2-methyl-2-butene, 3-methyl-1-butene, and/or cyclopentene), hexene (one or more of 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, methylcyclopentene and/or cyclohexene), heptenes, octenes, nonenes and decenes. The preference for specific olefins in the olefinic co-feed may depend on the purpose of the process, such as preferred production of ethylene or propylene.

In a preferred embodiment the olefinic co-feed preferably contains olefins having 4 or more carbon atoms (i.e. C₄+ olefins), such as butenes, pentenes, hexenes and heptenes. More preferably the olefinic fraction of the olefinic co-feed comprises at least 50 wt % of butenes and/or pentenes, even more preferably at least 50% wt of butenes, and most preferably at least 90 wt % of butenes. The butene may be 1-, 2-, or iso-butene, or a mixture of two or more thereof.

Preferably, at least part of the oxygenate feed is obtained converting methane into synthesis gas and proving the synthesis gas to an oxygenate synthesis zone to synthesise oxygenates. The methane is preferably obtained from natural gas or associated gas, more preferably the same natural gas or associated gas, from which the lower alkane feed was obtained.

Usually natural gas, comprises methane, ethane, propane, and butane in different quantities. Optionally, natural gas also comprises carbon dioxide. Preferably, a natural gas feed is first separated into at least a stream comprising predominantly methane and another stream comprising predominantly ethane, propane and/or butane. The first stream is used to prepare synthesis gas for synthesising an oxygenate feedstock to be supplied to step (b), while the later is used as lower alkane feed supplied to step (a).

In a further aspect, the invention provides an integrated system for producing olefins, which system comprises:

a) an alkane-to-aromatic system having one or more inlets for a lower alkane feed comprising at least one of ethane, propane and butane and an outlet for an alkane-to-aromatic zone effluent comprising hydrogen and aromatic conversion products including benzene;

b) an aromatic work-up system arranged to receive at least part of the alkane-to-aromatic zone effluent, the work-up section comprising a separation system for separating hydrogen from the alkane-to-aromatic effluent, an outlet for conversion products and an outlet for hydrogen;

c) an oxygenate-to-olefins conversion system, having one or more inlets for receiving an oxygenate feedstock, and comprising a reaction zone for contacting the oxygenate feedstock with an oxygenate conversion catalyst under oxygenate conversion conditions, and an outlet for an oxygenate-to-olefins zone effluent comprising ethylene;

d) an oxygenate synthesis system having one or more inlets for a feed containing carbon monoxide and/or carbon dioxide and an inlet for hydrogen, and an outlet for an oxygenate feedstock; and

means for providing hydrogen from the outlet for hydrogen of the workup section to the hydrogen inlet of the oxygenate synthesis system.

Optionally, the hydrogen ex. ATA is mixed with the feed containing carbon monoxide and/carbon dioxide prior to entering the oxygenate synthesis system. In that case, the inlets for feed containing carbon monoxide and/or carbon dioxide and an inlet for hydrogen of the oxygenate synthesis system may be the same inlet.

In a preferred embodiment the aromatic work-up system of the integrated system for producing olefins is additionally arranged to receive at least part of the fraction comprising aromatics, including at least benzene, from the OTO zone effluent.

Preferably, the system also comprises an ethylbenzene production unit, suitable for reacting benzene with ethylene. The ethylbenzene production unit comprising at least an inlet for conversion products from the work-up system, an inlet for ethylene from the oxygenate-to-olefins conversion system and an outlet for ethylbenzene. Preferably, the effluent from the oxygenates-to-olefins zone is first provided to ethylene workup section to separate ethylene from the remainder of the oxygenates-to-olefins zone effluent. Preferably, the system also comprises a catalytic dehydrogenation unit suitable for dehydrogenating ethylbenzene to at least styrene and hydrogen, comprising at least an inlet for ethylbenzene from the ethylbenzene production unit, an outlet for styrene and an outlet for hydrogen. Typically, the catalytic dehydrogenation unit will include a styrene workup section comprising a separation system for separating hydrogen from the styrene comprising effluent. More preferably, the system comprises in addition to a catalytic dehydrogenation unit, means for providing hydrogen from the outlet for hydrogen of the styrene workup section to the hydrogen inlet of the oxygenate synthesis system.

In an even further aspect, the invention provides a use of hydrogen obtained from a process to convert lower alkanes to aromatic hydrocarbons to produce an oxygenate feed for an oxygenate-to-olefin process.

### DETAILED DESCRIPTION OF THE DRAWINGS

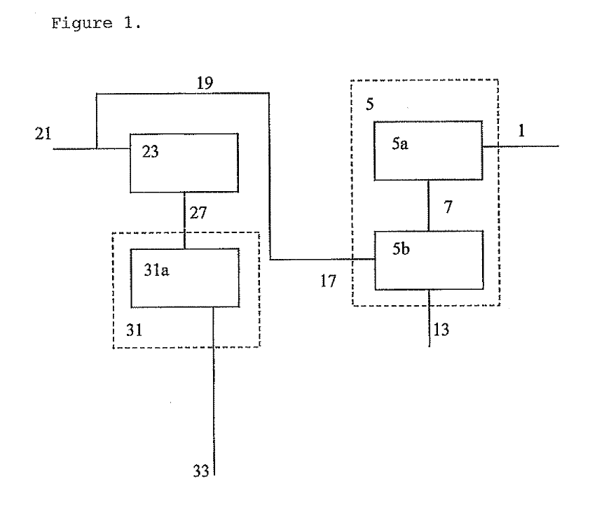

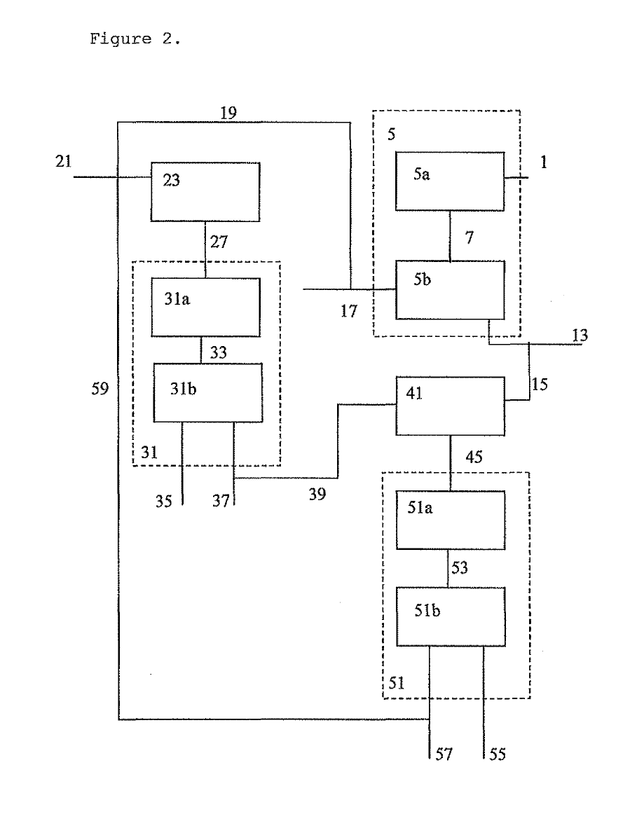

FIG. 1, a schematic representation is given of an embodiment of system for producing aromatic hydrocarbons and ethylene according to the invention. In the system of FIG. 1, a lower alkane feed comprising at least one of ethane, propane and butane is provided via conduit 1 to alkane-to-aromatic system 5, comprising alkane-to-aromatic zone 5a for converting ethane, propane and/or butane to at least benzene and hydrogen. Alkane-to-aromatic system 5 further comprises workup section 5b, which comprises at least a separation system to separate hydrogen from the alkane-to-aromatic zone effluent. From alkane-to-aromatic zone 5a, an alkane-to-aromatic zone effluent is retrieved via conduit 7 and provided via conduit 7 to aromatic workup section 5b, wherein the hydrogen is separated from the conversion products, including benzene. The conversion products are retrieved from aromatic workup section 5b via conduit 13, while the hydrogen is retrieved from workup section 5b via conduit 17. Optionally, a hydrodealkylation unit (not shown) is provided in alkane-to-aromatic system 5 to convert at least part of any toluene or xylenes in the alkane-to-aromatic zone effluent and/or the conversion products to benzene.

In FIG. 1, a feed containing carbon monoxide and/or carbon dioxide, e.g. a synthesis gas is provided via conduit 21 to oxygenate synthesis system 23, comprising an oxygenate synthesis zone for synthesizing oxygenates from hydrogen and at least one of carbon monoxide and carbon dioxide. From oxygenate synthesis system 23, an oxygenate feedstock is retrieved via conduit 27. The oxygenate feedstock is provided to oxygenate-to-olefins conversion system 31, comprising oxygenate-to-olefins zone 31a for converting oxygenates to at least ethylene. Optionally, an olefinic co-feed (not shown) is provided to oxygenate-to-olefins conversion system 31 together with the oxygenate feedstock. From oxygenate-to-olefins conversion system 31, an oxygenate-to-olefins zone effluent is retrieved via conduit 33, which comprises at least ethylene.

As mentioned hydrogen is retrieved from workup section 5b of alkane-to-aromatic system 5 via conduit 17. At least part of the hydrogen in conduit 17 is provided via conduit 19 to conduit 21 and mixed with the synthesis gas, i.e. feed containing carbon monoxide and/or carbon dioxide.

In FIG. 2, a further schematic representation is given of an embodiment of a system according to the invention. In FIG. 2, the system of FIG. 1 is expanded by providing ethylbenzene production unit 41. At least part of the conversion products retrieved from workup section 5b via conduit 13 is provided via conduit 15 to ethylbenzene production unit 41.

The effluent from oxygenate-to-olefins conversion system 31 may comprise other olefins, such as propylene, but also unreacted oxygenates, such as methanol or DME, paraffins or other unsaturates. Therefore, preferably, oxygenate-to-olefins conversion system 31 also comprises a ethylene workup section 31b. The oxygenate-to-olefins zone effluent in conduit 33 is first provided to ethylene workup section 31b, wherein ethylene is separated from the remainder of the oxygenate-to-olefins zone effluent. The remainder of the oxygenate-to-olefins zone effluent is retrieved from oxygenate-to-olefins conversion system 31 via conduit 35. The ethylene is retrieved from the oxygenate-to-olefins conversion system 31 via conduit 37. At least part of the ethylene retrieved from oxygenate-to-olefins conversion system 31 in conduit 37 is provided to ethylbenzene production unit 41 via conduit 39.

In ethylbenzene production unit 41, benzene is reacted with ethylene to form ethylbenzene. The ethylbenzene is retrieved from ethylbenzene production unit 41 via conduit 45 and provided to dehydrogenation unit 51. In dehydrogenation unit 51, ethylbenzene is catalytically dehydrogenation unit 51 in the presence of steam to at least styrene monomer and hydrogen. If insufficient steam is present in dehydrogenation unit 51 additional water of steam may be provided via a separate conduit (not shown) to dehydrogenation unit 51. Preferably, dehydrogenation unit 51 comprises catalytic dehydrogenation zone 51a and styrene workup section 51b, comprising separation means to separate the hydrogen from the styrene monomer. The effluent of dehydrogenation zone 51a is provided to styrene workup section 51b via conduit 53. Styrene monomer is retrieved from dehydrogenation unit 51 via conduit 55. Hydrogen is retrieved from dehydrogenation unit 51 via conduit 59. At least part of the hydrogen in conduit 57 is provided via conduit 59 to conduit 21 and mixed with the synthesis gas, i.e. feed containing carbon monoxide and/or carbon dioxide.

### EXAMPLES

The invention is illustrated by the following non-limiting calculated examples.

### Example 1

In the Examples, several options of implementing the present invention are compared with comparative examples, by means of model calculations. As basis for Examples 1a to g, a model integrated OTO/ATA process was taken. In Table 1, an overview is provided of the feed input and the calculated products.

Calculations were done using proprietary models for modelling the OTO and ATA conversion. The key input to the models was as follows:

### ATA Conversion:

A feed containing 31.6 wt % ethane, 29.5 wt % propane and 38.9 wt % butane is converted to at least benzene, toluene and xylenes, using a two stage reactor system. 3300 t/d of mixed feed are fed to a first stage aromatization reactor that uses a catalyst for aromatizing these lower alkanes. The first stage reactor operates at about 1 atmosphere pressure and at a temperature of about 600° C.

The first stage reactor effluent is then mixed with the reactor effluent from the second stage reactor described below. The combined effluent from both the reactor stages is then fed to a separation system where unconverted reactants and light hydrocarbons that consist primarily of ethane and some other hydrocarbons, which may include ethylene, propane, propylene, methane, butane and some hydrogen, are isolated and used as the feed for the second stage aromatization reactor, which uses a catalyst for aromatizing these lower alkanes.

The second stage reactor operates at about 1 atmosphere pressure and a temperature of about 620° C. The effluent from the second stage reactor is mixed with the effluent from the first stage reactor as described above. The gas hourly space velocity (GSVH) in both stages is 1000 hr⁻¹.

The average single pass conversion for the mixed feed is obtained from the cumulative conversion of ethane, propane and butane in the feed over both the stages and is calculated to be 74.5 mol %, based on the moles of ethane, propane and butane in the feed. Overall BTX (benzene, toluene and xylenes) yield is 62.6 wt %, based on the weight of the feed. Almost 10 wt % of the mixed feed is converted into C9+ hydrocarbons. Hydrogen is produced at a yield of 8.4 wt % of the mixed feed. The balance is fuel gas.

### OTO Conversion:

5012 t/d MeOH is fed to the OTO reactor together with 1384 t/d of recycled and superheated steam and 1520 t/d of recycled C4 stream. The model was calibrated on small-scale experiments conducted to determine product distributions for single-pass OTO conversions. Therein, all components that were fed to the OTO reactor have been evaporated and heated such that the temperature in the reactor is controlled between 550-600° C. The pressure in the reactor is 2 bar absolute. The OTO catalyst is fluidized in the reaction medium under the condition that the weight hourly space velocity (WHSV) is 4-10 h⁻¹, whereby WHSV is defined as the total weight of the feed flow over the catalyst weight per hour. The following catalyst was used: Composition and preparation: 32 wt % ZSM-23 SAR 46, 8 wt % ZSM-5 SAR 280, 36 wt % kaolin, 24 wt % silica sol, and, after calcination of the ammonium form of the spray dried particle, 1.5 wt % P was introduced by H₃PO₄ impregnation. The catalyst was again calcined at 550 C. The steam and C4 recycle streams are excluded from the product composition tables.

The methanol provided to the OTO process (approximately 5000 t/d, see Table 1) is synthesised using at least part of the hydrogen ex. ATA.

The feed containing carbon monoxide and/or carbon dioxide is synthesis gas. The synthesis gas may be obtained from any type of synthesis gas producing processes or is prepared by combining synthesis gas obtained from one or more synthesis gas producing processes and optionally a carbon dioxide stream obtained from a subsurface gas reservoir, i.e. field carbon dioxide, however this carbon dioxide can also be for instance be carbon oxide obtained from a MEG synthesis process. The yields of methanol are calculated by an Aspen model. To keep the inert concentration of about 40 wt % in the synthesis gas recycle, the amount of purge stream from the recycle is adjusted.

### Styrene Monomer Conversion

Benzene obtained from the ATA step (a) and ethylene obtained from the OMO step (b) are reacted to form ethylbenzene. The obtained ethylbenzene is dehydrogenated to styrene monomer and hydrogen. For the ease of calculation it has been assumed that full conversion is achieved and pure ethylbenzene and, respectively, pure styrene monomer and hydrogen (further referred to as hydrogen ex. styrene) are obtained.

The hydrogen ex. ATA, hydrogen ex. styrene and carbon dioxide ex. field are taken as 99.9+% pure.

Natural gas composition is 94.3 mol % CH₄, 0.6 mol % C₂H₆, 4.6 mol % N₂, 0.4 mol % CO₂, and 0.1 mol % Ar, based on the total number of moles in the natural gas stream.

The Used Synthesis Gases were:

Synthesis gas from a non-catalytic partial oxidation of natural gas (Shell gasification process). The SGP syngas comprised 61.2 mol % hydrogen, 34.0 mol % carbon monoxide, 2.1 mol % carbon dioxide and 2.5 mol % inert (N₂, Ar and CH₄), based on the total number of moles in the SGP syngas.

Synthesis gas from an auto-thermal reforming of natural gas (ATR). The ATR syngas comprised 65.5 mol % hydrogen, 26.7 mol % carbon monoxide, 6.4 mol % carbon dioxide and 1.7 mol % inert (N₂, Ar and CH₄), based on the total number of moles in the ATR syngas.

A mixture of synthesis gas from a steam methane reforming (SMR) and a SGP synthesis gas. The mixture comprised 65.8 mol % hydrogen, 25.6 mol % carbon monoxide, 4.4 mol % carbon dioxide and 3.8 mol % inert (N₂, Ar and CH₄), based on the total number of moles in the mixture of syngas.

Table 2a provides an overview of the feed, i.e. hydrogen ex. ATA and the feed containing carbon monoxide and/or carbon dioxide, provided to the methanol synthesis.

Table 2b provides an overview of the feed composition provided to the methanol synthesis.

Table 3 provides an overview of the feedstock, i.e. natural gas, oxygen and water, required to produce the synthesis gas.

Table 4 shows the methanol production based on waste carbon dioxide.

Experiment 1a: (not according to the invention)

The methanol feed to the OTO process is synthesised from a mixture of SGP and SMR synthesis gas. 2949 ton/day of natural gas is required produce sufficient methanol. Experiment 1b:

The methanol feed to the OTO process is synthesised from a mixture of part of the hydrogen ex. ATA and SGP synthesis gas. By providing hydrogen ex. ATA to the methanol synthesis, the natural gas consumption for producing the methanol has decreased by 8 wt % based on the natural gas required for producing the methanol in Experiment 1a. There is no longer the need to add additional SMR synthesis gas. Moreover, by not using a SMR synthesis gas the water consumption decreases significantly, in principle no water is used for the synthesis gas production.

Furthermore, inert (N₂, Ar and CH₄) concentration in the feed to the methanol synthesis are reduced, compared to the levels seen in experiment 1a, due to dilution of the SGP synthesis gas with hydrogen obtained from the ATA process.

### Experiment 1c:

The methanol feed to the OTO process is synthesised from a mixture of part of the hydrogen ex. ATA and SGP synthesis gas. In addition, pure carbon dioxide ex. field is added to increase the carbon dioxide content to 3.3 mol %, based on the total feed to the methanol synthesis. The natural gas consumption for producing the methanol has decreased by 12 wt % based on the natural gas required for producing the methanol in Experiment 1a. In addition, 255 ton/day of methanol is produced based on waste carbon dioxide, i.e. carbon dioxide not produced as part of the process to prepare synthesis gas, which would need to be sequestered or otherwise captured and stored. As a result, the carbon dioxide penalty of the process is reduced.

Again, inert (N₂, Ar and CH₄) concentrations are further lowered.

### Experiment 1d:

The methanol feed to the OTO process is synthesised from a mixture of all of the hydrogen ex. ATA, SGP synthesis gas and the addition of additional hydrogen ex styrene production unit. In addition, pure carbon dioxide ex. field is added to increase the carbon dioxide content to 7.9 mol %, based on the total feed to the methanol synthesis. The natural gas consumption for producing the methanol has decreased by 27 wt % based on the natural gas required for producing the methanol in Experiment 1a. In addition 1062 ton/day of methanol is produced based on waste carbon dioxide.

Again, inert (N₂, Ar and CH₄) concentrations are further lowered.

### Experiment 1e:

The methanol feed to the OTO process is synthesised from a mixture of part of the hydrogen ex. ATA and ATR synthesis gas. By providing hydrogen ex. ATA to the methanol synthesis, the natural gas consumption decrease as compared Experiment 1a. The natural gas consumption for producing the methanol has decreased by 1 wt % based on the natural gas required for producing the methanol in Experiment 1a. There is no longer the need to add additional SMR synthesis gas.

Furthermore, inert (N₂, Ar and CH₄) concentration in the feed to the methanol synthesis are reduced, compared to the levels seen in experiment 1a, due to dilution of the ATR synthesis gas with hydrogen obtained from the ATA process step.

### Experiment 1f:

The methanol feed to the OTO process is synthesised from a mixture of part of the hydrogen ex. ATA and ATR synthesis gas. In addition, pure carbon dioxide ex. field is added to increase the carbon dioxide content to 7.1 mol %, based on the total feed to the methanol synthesis. The natural gas consumption for producing the methanol has decreased by 6 wt % based on the natural gas required for producing the methanol in Experiment 1a. In addition 273 ton/day of methanol is produced based on waste carbon dioxide. As a result, the carbon dioxide penalty of the process is reduced.

Again, inert (N₂, Ar and CH₄) concentrations are further lowered.

### Experiment 1g:

The methanol feed to the OTO process is synthesised from a mixture of part of the hydrogen ex. ATA and ATR synthesis gas. In addition, pure carbon dioxide ex. field is added to increase the carbon dioxide content to 7.9 mol %, based on the total feed to the methanol synthesis. The natural gas consumption for producing the methanol has decreased by 9 wt % based on the natural gas required for producing the methanol in Experiment 1a. In addition 443 ton/day of methanol is produced based on waste carbon dioxide. As a result, the carbon dioxide penalty of the process is reduced.

Again, inert (N₂, Ar and CH₄) concentrations are further lowered.

TABLE 1

| | | | Integrated | Integrated |
|---|---|---|---|---|
| | OTO | ATA | OTO/ATA* | OTO/ATA/styrene |
| | 10³ kg/day | 10³ kg/day | 10³ kg/day | 10³ kg/day |
| Feed: | | | | |
| Methanol | 5012 | | 5012 | 5012 |
| Ethane | | 1043 | 1026 | 1026 |
| Propane | | 974 | 926 | 926 |
| Butane | | 1284 | 1284 | 1284 |
| Products: | | | | |
| Ethylene | 512 | | 512 | 90 |
| Propylene | 1275 | | 1275 | 1275 |
| Benzene | | 1175 | 1175 | |
| Toluene | | 739 | 739 | 820 |
| Xylenes | | 158 | 158 | 176 |
| Styrene | | | | 1566 |
| monomer | | | | |
| Ethane | 17 | | | |
| propane | 48 | | | |
| Other^{#} | 318 | 330 | 648 | 648 |
| Fuel gas | 98 | 620 | 718 | 718 |
| H₂O | 2710 | | 2710 | 2710 |
| Hydrogen | 17 | 277 | 294 | 324 |

*including recycling ethane and propane in the effluent of the OTO to the ATA.
^{#}including C3+ components from the OTO process step and C9+ components from the ATA process step.

TABLE 2a

| | Feed | | | Product |
|---|---|---|---|---|
| | 10³ kg/day | | | 10³ kg/day |
| Exp. | H₂ ex. ATA | Synthesis gas | CO₂ ex. field | Methanol |
| 1a | — | 2236 (ex.SMR) | — | 5000 |
| | | 3883 (ex.SGP) | | |
| 1b | 129 | 5488 (ex.SGP) | — | 5000 |
| 1c | 173 | 5208 (ex.SGP) | 365 | 5000 |
| 1d | 321 | 4322 (ex.SGP) | 1584 | 5000 |
| 1e | 82 | 5837 (ex.ATR) | — | 5000 |
| 1f | 134 | 5518 (ex.ATR) | 398 | 5000 |
| 1g | 165 | 5320 (ex.ATR) | 635 | 5000 |

TABLE 2b

| | Composition mol %* | | | | | Molar |
|---|---|---|---|---|---|---|
| Exp. | H₂ | CO | CO₂ | N₂, AR, CH₄ | H₂O | ratio |
| 1a | 65.8 | 25.6 | 4.4 | 3.8 | 0.2 | 2.05 |
| 1b | 66.1 | 29.7 | 1.8 | 2.2 | 0.2 | 2.04 |
| 1c | 66.7 | 27.8 | 3.3 | 2.0 | 0.2 | 2.04 |
| 1d | 68.5 | 21.8 | 8.0 | 1.6 | 0.1 | 2.03 |
| 1e | 68.1 | 24.6 | 5.9 | 1.4 | >0.1 | 2.04 |
| 1f | 68.6 | 22.9 | 7.2 | 1.3 | >0.1 | 2.04 |
| 1g | 69.0 | 21.9 | 7.9 | 1.2 | >0.1 | 2.05 |

*Based on the total number of moles in the feed
**molar ratio = (#mol H₂ − #mol CO₂)/(#mol CO + #mol CO₂)

TABLE 3

| | Feed | | | Total |
|---|---|---|---|---|
| | 10³ kg/day | | | feedstock |
| Exp. | Natural gas | O₂ | H₂O | 10³ kg/day |
| 1a | 2949 | 2291 | 1210 | 6450 |
| 1b | 2722 | 3239 | 0* | 5961 |
| 1c | 2583 | 3074 | 0* | 5657 |
| 1d | 2144 | 2551 | 0* | 4695 |
| 1e | 2933 | 3109 | 1691 | 7733 |
| 1f | 2773 | 2939 | 1599 | 7311 |
| 1g | 2674 | 2834 | 1541 | 7049 |

*In principle no water is added.

TABLE 4

| | Feed | Product | |
|---|---|---|---|
| | 10³ kg/day | 10³ kg/day | |
| Exp. | CO₂ ex field | Methanol | Methanol from CO₂ ex. field |
| 1a | — | 5000 | |
| 1b | — | 5000 | |
| 1c | 365 | 5000 | 255 |
| 1d | 1584 | 5000 | 1062 |
| 1e | — | 5000 | |
| 1f | 398 | 5000 | 273 |
| 1g | 635 | 5000 | 443 |

By integrating the OTO and ATA process, the feed requirements of the ATA are partially fulfilled by recycling the ethane and propane produced by the OTO process to the ATA process. As a result less C2 to C4 paraffins need to be provided to the ATA process to produce the same amount of benzene, toluene and xylenes. Although not shown in the experiments above, the OTO process also produces C4 paraffins. These may also be recycled to the ATA process further reducing the amount of C2 to C4 paraffins that need to be provided to the process.

The benzene, ethyl benzene and/or styrene monomer output can be further increased by converting at least part of the toluene and xylenes to benzene and optionally to benzene and/or styrene monomer as described herein above. The toluene and xylenes may be converted to benzene for instance by a hydrodealkylation process in the presence of hydrogen. The hydrogen can be provide externally or may be hydrogen produced by in the ATA process itself.

By integrating an OTO process with an ATA process according to the present invention and providing at least part of the hydrogen produced during the ATA process to the OTO process to form part of the feed to produce the oxygenate, it is possible to capture significant amounts of waste carbon dioxide in the form of methanol, ethylene and/or styrene monomer and products derived thereof. In addition, by using a synthesis gas such as SGP synthesis gas, which comprises relatively low amounts of carbon dioxide it is possible to capture even more waste carbon dioxide in the form of methanol, ethylene and/or styrene monomer and products derived thereof. In addition, by using a synthesis gas with is produced by a process in which in principle no or only little water is used, such as a non-catalytic partial oxidation process, water consumption is significantly lowered.

As can be seen from table 1, by integrating an OTO process with an ATA process according to the present invention it is possible to significantly reduce the amount of gaseous ethylene that is produced and instead produce ethylbenzene and/or styrene monomer, which are both liquids under ambient conditions. These liquids products can be stored and transported, without the need for high pressure and/or low temperature storage or transporting means.

The remaining ethylene can be converted, via ethylene oxide, to MEG and carbon dioxide providing an additional source of carbon dioxide as well as further reducing the ethylene output.

1. A process for producing aromatic hydrocarbons and ethylene, comprising:
a. contacting a lower alkane feed comprising at least one of ethane, propane and butane with an aromatic hydrocarbon conversion catalyst within an alkane-to-aromatic zone to obtain at least hydrogen and aromatic reaction products, including at least benzene;
b. converting an oxygenate feedstock in an oxygenate-to-olefin zone to obtain olefins, including at least ethylene;
wherein at least part of the oxygenate feedstock is obtained by providing at least part of the hydrogen obtained in step a) and a feed containing carbon monoxide and/or carbon dioxide to an oxygenate synthesis zone and synthesizing oxygenates.

2. A process according to claim 1, wherein the process is a process for preparing ethylbenzene, further comprising the step:
c. reacting at least part of the benzene obtained in step (a) with at least part of the ethylene obtained in step (b) to obtain ethylbenzene.

3. A process according to claim 2, wherein the process is a process for preparing styrene monomer, further comprising the step:
d. dehydrogenating at least part of the ethylbenzene obtained in step (c) to obtain styrene monomer and hydrogen; and providing at least part of the hydrogen obtained in step d) to the oxygenate synthesis zone to synthesize oxygenates.

4. A process according to claim 1, wherein methane is obtained from step (a) and at least part of the feed containing carbon monoxide and/or carbon dioxide is obtained by converting at least part of the methane to hydrogen and at least one of carbon monoxide and carbon dioxide.

5. A process according to claim 1, wherein the aromatic conversion products obtained in step (a) further include toluene and/or xylene and at least part of the toluene and/or xylene is hydrodealkylated in the presence of hydrogen to obtain further benzene and methane.

6. A process according to claim 1, comprising providing hydrogen and at least one of carbon monoxide and carbon dioxide to the oxygenate synthesis zone in a molar ratio of in the range of from 2.0 to 3.0.

7. A process according to claim 1, wherein the feed containing carbon monoxide and/or carbon dioxide comprises a synthesis, preferably synthesis gas with a molar ratio of hydrogen to carbon monoxide and/or carbon dioxide of in the range of from 1.0 to 1.9.

8. A process according to claim 1, wherein the feed containing carbon monoxide and/or carbon dioxide comprises carbon dioxide obtained from:
i) a subsurface natural gas or oil reservoir; and/or
ii) a process for preparing ethylene oxide and Mono-ethylene-glycol.

9. A process according to according to claim 1, further comprising providing at least part of the ethylene obtained in step (b) to an ethylene oxidation zone together with a source of oxygen and performing a process for preparing ethylene oxide to obtain ethylene oxide and carbon dioxide.

10. A process according to claim 1, comprising:
i) providing a feed comprising methane and at least one of ethane, propane and butane;
ii) separating the feed into at least a methane-comprising feed and a lower alkane feed, comprising at least one of ethane, propane and butane;
iii) providing at least part the methane-comprising feed to a process for preparing synthesis gas to obtain a synthesis gas; and
iv) contacting the lower alkane feed with an aromatic hydrocarbon conversion catalyst in an alkane-to-aromatic zone according to step (a) to obtain at least benzene and hydrogen;
v) providing at least part of the hydrogen obtained in step iv) and at least part the synthesis gas obtained in step iii) to an oxygenate synthesis zone and synthesising oxygenates; and
vi) converting at least part of the oxygenates in an oxygenate-to-olefin zone according to step (b) to obtain at least ethylene.

11. A process according to claim 10, wherein the feed comprising methane and at least one of ethane, propane and butane is natural gas or associated gas.

12. (canceled)

13. An integrated system for producing olefins, which system comprises:
a) an alkane-to-aromatic system having one or more inlets for a lower alkane feed comprising at least one of ethane, propane and butane and an outlet for an alkane-to-aromatic effluent comprising hydrogen and aromatic conversion products including benzene;
b) an aromatic work-up system arranged to receive at least part of the alkane-to-aromatic effluent, the work-up section comprising a separation system for separating hydrogen from the alkane-to-aromatic effluent, an outlet for conversion products and an outlet for hydrogen;
c) an oxygenate-to-olefins conversion system, having one or more inlets for receiving an oxygenate feedstock, and comprising a reaction zone for contacting the oxygenate feedstock with an oxygenate conversion catalyst under oxygenate conversion conditions, and an outlet for an oxygenate-to-olefins effluent comprising ethylene;
d) an oxygenate synthesis system having one or more inlets for a feed containing carbon monoxide and/or carbon dioxide and an inlet for hydrogen, and an outlet for an oxygenate feedstock; and
means for providing hydrogen from the outlet for hydrogen of the workup section to the hydrogen inlet of the oxygenate synthesis system.
